(19) United States
(12) Patent Application Publication
(10) Pub. No.: US 2007/0238708 A1
(43) Pub. Date: Oct. 11, 2007

(54) Acute Inflammatory Condition Treatment

(75) Inventors: Arkady Mandel, Mississauga (CA); Anthony Bolton, Dublin (IE)

Correspondence Address: FOLEY & LARDNER LLP, 1530 PAGE MILL ROAD, PALO ALTO, CA, 94304, US

(73) Assignee: Vasogen Ireland Limited, Shannon, County Clare (IE)

(21) Appl. No.: 10/565,360

(22) PCT Filed: Jul. 20, 2004
(86) PCT No.: PCT/CA04/01053

§ 371 Date: Feb. 26, 2007

Related U.S. Application Data
(60) Provisional application No. 60/489,071, filed on Jul. 21, 2003.

(51) Int. Cl. A61K 31/6615

(52) U.S. Cl. 514/121

(57) ABSTRACT

This invention provides a method for prophylaxis or treatment of an acute inflammatory disorder, comprising administering to a patient an effective amount of pharmaceutically acceptable bodies carrying an effective number of phosphate-containing groups presented or presentable on the surface of said bodies, the phosphate-containing groups comprising a plurality of phosphate-glycerol groups or groups convertible to such groups, to inhibit and/or reduce the progression of the acute inflammatory disorder, said bodies being of a size from about 20 nanometers (nm) to 500 micrometers (μm).

### FIELD OF THE INVENTION

This invention relates to processes and compositions for alleviating acute inflammatory conditions in mammalian patients.

### BACKGROUND OF THE INVENTION

“Acute inflammatory conditions” as the term is used herein, and in accordance with normal medical parlance, refers to inflammatory conditions having a rapid onset and severe symptoms. The duration of the onset, from a normal condition of the patient to one in which symptoms of inflammation are seriously manifested, is anything up to about 72 hours. Acute inflammatory conditions are to be contrasted with chronic inflammatory conditions, which are inflammatory conditions of long duration, denoting a disease showing little change or of slow progression. The distinction between acute and chronic conditions is well known to those in the medical professions, even if they are not distinguishable by rigid, numbers-based definitions.

It is known that many inflammatory conditions are associated with an abnormal secretion level of various cytokines in the mammalian body. Professional antigen-presenting cells (APCs), including dendritic cells and macrophages, actively capture and process antigens, clear cell debris, and remove infectious organisms and dying cells, including the residues from dying cells. During this process, APCs can stimulate the production of either inflammatory Th 1 pro-inflammatory cytokines (IL-12, IL-1, INF-α, IFN-γ, etc); or regulatory, Th2/Th3 anti-inflammatory cytokines (IL-10, IL-4, TGF-β etc) dominated responses; depending on the nature of the antigen or phagocytosed material and the level of APC maturation/activation.

### SUMMARY OF THE INVENTION

The present invention is based upon the discovery that pharmaceutically acceptable bodies, such as liposomes, beads or similar particles, which present phosphate-glycerol head groups, will, upon administration to a mammalian patient, cause a rapid increase in the level of anti-inflammatory cytokines such as TGF-β and/or conversely a rapid decrease in the level of inflammatory cytokines such as TNF-α, IFN-γ and IL-12, the effects being significant within the first twelve hours after the administration of the bodies. Accordingly, they may be used to treat acute inflammatory diseases and/or delaying and/or ameliorating symptoms associated with such diseases.

In a preferred embodiment, the invention is directed to a process of producing a rapid anti-inflammatory response in a mammalian patient, as evidenced by altered cytokine profiles, comprising administering to the patient a composition of matter including pharmaceutically acceptable bodies of a size from about 20 nanometers (nm) to 500 micrometers (μm), the bodies carrying an effective number of phosphate containing groups accessible for interaction or reaction such as being presented or presentable on the surface of the bodies. The phosphate containing groups comprise a plurality of phosphate-glycerol groups or groups convertible to such groups. Preferably, the bodies are essentially free of pharmaceutically active entities other than phosphate containing groups. Following administration to a mammal, the bodies, through the phosphate-glycerol groups, are believed to interact rapidly with the immune system resulting in the rapid development of an anti-inflammatory response, as evidenced by changes in cytokine profile.

In one aspect this invention provides, a method for prophylaxis or treatment of an acute inflammatory disorder comprising administering to a patient an effective amount of pharmaceutically acceptable bodies carrying an effective number of phosphate-containing groups presented or presentable on the surface of said bodies, the phosphate-containing groups comprising a plurality of phosphate-glycerol groups or groups convertible to such groups, to inhibit and/or reduce the progression of the acute inflammatory disorder, said bodies being of a size from about 20 nanometers (nm) to 500 micrometers (μm).

This invention is further directed to a method for treating an acute inflammatory disorder comprising administering to a patient an effective amount of pharmaceutically acceptable bodies carrying an effective number of phosphate-glycerol groups or groups convertible to such groups, to inhibit and/or reduce the progression of the acute inflammatory disorder, said bodies being of a size from about 20 nanometers (nm) to 500 micrometers (μm), comprising a plurality of phosphate-glycerol groups.

Optionally, the bodies described above may additionally comprise an inactive constituent surface group, and/or a constituent surface group such as another phosphate containing group, which is active through another mechanism,.e.g. phosphatidylserine. (See, e.g. Fadok et al., International Publication WO 01/66785). Such constituent surface groups, if present, should not constitute more than about 40% of the total of functional surface groups, balance phosphate glycerol.

In another aspect, this invention provides use of pharmaceutically acceptable bodies carrying an effective number of phosphate-glycerol groups or groups convertible to phosphate-glycerol groups, to inhibit and/or reduce the progression of the acute inflammatory disorder, said bodies being of a size from about 20 nanometers (nm) to about 500 micrometers (μm), in the preparation of a medicament for the treatment of an acute inflammatory disorder.

### BRIEF DESCRIPTION OF THE DRAWINGS

The accompanying Figures are graphical presentation of the results of Example 1 below, DNFB induced contact inflammatory response model in mice experiments using liposomes, in accordance with a preferred embodiment of the invention. More specifically:

FIG. 1 is a graph of TNFα cytokine production in lymph nodes of the animals, against time;

FIG. 2 is a similar graph for the cytokine IFN-γ;

FIG. 3 is a similar graph for the cytokine TGF-β;

FIG. 4 is a similar graph for the cytokine IL-12;

FIG. 5 is a graphical presentation of TNFα concentration from macrophages, Example 2 herein;

FIG. 5A is a similar graphical presentation of the comparative experiments detailed in Example 2;

FIG. 6 is a graphical presentation of the IL-4 concentration of hippocampal IL-4 concentration from rats treated according to Example 3;

FIG. 7 is a similar graphical presentation of IFN-γ concentrations in serum of rats treated according to Example 4.

### DESCRIPTION OF PREFERRED EMBODIMENTS

According to the present invention, pharmaceutically acceptable bodies carrying phosphate-glycerol groups on their surface are administered to patients suffering from acute inflammatory disorders with increased levels of inflammatory cytokines and/or decreased levels of anti-inflammatory cytokines.

The preferred pharmaceutically acceptable bodies for use in the process of the present invention include synthetic and semi-synthetic bodies having shapes which are typically but not exclusively spheroidal, cylindrical, ellipsoidal, including oblate and prolate spheroidal, serpentine, reniform etc., and sizes from about 20 nanometres to about 500 μm in diameter, preferably measured along its longest axis, and comprising phosphate-glycerol groups on the surface thereof. Such synthetic and semi-synthetic bodies are disclosed below and also found in, for example, Bolton et al., U.S. Ser. No. 10/348,600 and U.S. Ser. No. 10/348,601, herein incorporated in their entirety by reference.

The pharmaceutically acceptable bodies have phosphate-glycerol groups of predetermined characteristics on the exterior surface. Without being limited to any one theory, it is believed that these groups are capable of interacting with the appropriate receptor(s), other than exclusively the PS receptor, on antigen presenting cells in vivo. The structure of these groups may be synthetically altered and include all, part of or a modified version of the original phosphate-glycerol group. For example, the negatively charged oxygen of the phosphate group of the phosphate-glycerol group may be converted to a phosphate ester head group (e.g., L-OP(O)(OR′)(OR′″), where L is the lipid-glycerol remainder of the phospholipid described below, R′ is —CH₂CH(OH)CH₂OH and R′″ is alkyl of from 1 to 4 carbon atoms or hydroxyl substituted alkyl of from 2 to 4 carbon atoms, and 1 to 3 hydroxyl groups provided that R′″ is more readily hydrolyzed in vivo than the R′ group; to a diphosphate group including diphosphate esters (e.g., L-QP(O)(OR′)OP(O)(OR″)₂ wherein L and R′ are as defined above and each R″ is independently hydrogen, alkyl of from 1 to 4 carbon atoms, or a hydroxyl substituted alkyl of from 2 to 4 carbon atoms and 1 to 3 hydroxyl groups provided that the second phosphate group [—P(O)(OR″)₂] is more readily hydrolyzed in vivo that the R′ group; or to a triphosphate group including triphosphate esters (e.g., L-OP(O)(OR′)OP(O)(OR″)OP(O)(OR″)₂ wherein L and R′ are defined as above and each R″ is independently hydrogen, alkyl of from 1 to 4 carbon atoms, or a hydroxyl substituted alkyl of from 2 to 4 carbon atoms and 1 to 3 hydroxyl groups provided that the second and third phosphate groups are more readily hydrolyzed in vivo than the R′ group; and the like. Such synthetically altered phosphate-glycerol groups are capable of expressing phosphate-glycerol in vivo and, accordingly, such altered groups are phosphate-glycerol convertible groups.

Phosphatidylglycerol is a known compound. It can be produced, for example, by treating the naturally occurring dimeric form of PG, cardiolipin, with phospholipase D. It can also be prepared by enzymatic synthesis from phosphatidylcholine using phospholipase D—see, for example, U.S. Pat. No. 5,188,951 Tremblay, et al. Chemically, it has a phosphate-glycerol head group and a pair of similar but different C₁₈-C₂₀ fatty acid chains.

As used herein the term “PG” is intended to cover phospholipids carrying the phosphate-glycerol group with a wide range of at least one fatty acid chains provided that the resulting PG entity can participate as a structural component of a liposome. Preferably, such PG compounds can be represented by the Formula I: 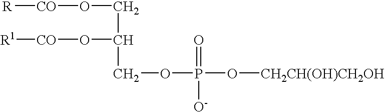
where R and R¹ are independently selected from C₁-C₂₄ hydrocarbon chains, saturated or unsaturated, straight chain or containing a limited amount of branching wherein at least one chain has from 10 to 24 carbon atoms. Essentially, the lipid chains R and R¹ form the structural component of the liposomes, rather than the active component. Accordingly, these can be varied to include two or one such lipid chains, the same or different, provided they fulfill the structural function. Preferably, the lipid chains may be from about 10 to about 24 carbon atoms in length, saturated, mono-unsaturated or polyunsaturated, straight-chain or with a limited amount of branching. Laurate (C12), myristate (C14), palmitate (C16), stearate (C18), arachidate (C20), behenate (C22) and lignocerate (C24) are examples of useful saturated lipid chains for the PG for use in the present invention. Palmitoleate (C16), oleate (C18) are examples of suitable mono-unsaturated lipid chains. Linoleate (C18), linolenate (C18) and arichidonate (C20) are examples of suitable poly-unsaturated lipid chains for use in PG in the liposomes of the present invention. Phospholipids with a single such lipid chain, also useful in the present invention, are known as lysophospholipids.

The present invention also extends to cover use of liposomes in which the active component is the dimeric form of PG, namely cardiolipin but other dimers of Formula I are also suitable. Preferably, such dimers are not synthetically cross-linked with a synthetic cross-linking agent, such as maleimide but rather are cross-linked by removal of a glycerol unit as described by Lehniger, Biochemistry, p. 525 (1970) and depicted in the reaction below: 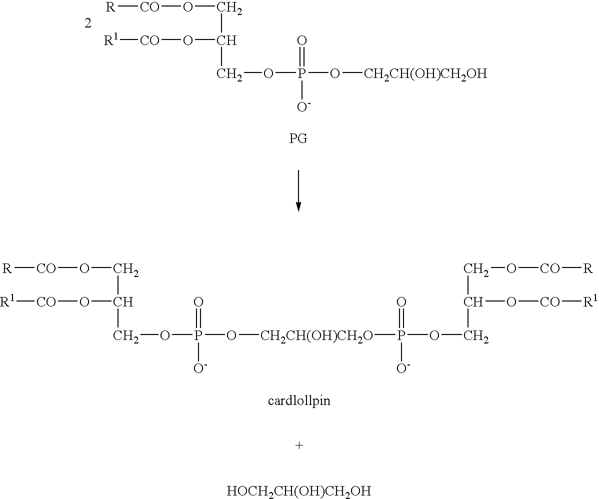
where each R and R¹ are independently as defined above.

As noted above and again without being limited to any one theory, the PG group and its dimer are believed to be a ligand since it is believed that it binds to a specific site on a protein or other molecule (“PG receptor”) and, accordingly, this molecule of phosphatidylglycerol (and its dimeric form) is sometimes referred to herein as a “ligand” or a “binding group.” Such binding is believed to take place through the phosphate-glycerol group —O—P(═O(OH)—O—CH₂—CH(OH)—CH₂—OH, which is sometimes referred to herein as the “head group,” “active group,” or “binding group.” In view of the above, reference to “binding,” “binding group,” or “ligand” herein is not to infer any mechanism or mode of action. Nevertheless, it is believed that the above phosphate-glycerol head groups are presented on the exterior surfaces of the bodies of the present invention for rapid interaction with components of the patient's immune system. This interaction, it should be noted, is not the same as the specific interaction of apoptotic cells with the phosphatidylserine receptor on antigen presenting cells.

Examples of “three-dimensional body portions” or pharmaceutically acceptable bodies” include biocompatible synthetic or semi-synthetic entities such as liposomes, solid beads, hollow beads, filled beads, particles, granules and microspheres of biocompatible materials, natural or synthetic, as commonly used in the pharmaceutical industry. The beads may be solid or hollow, or filled with biocompatible material. The term “biocompatible” refers to substances which in the amount employed are either non-toxic or have acceptable toxicity profiles such that their use in vivo is acceptable. Likewise the term “pharmaceutically acceptable” as used in relation to “pharmaceutically acceptable bodies” refers to bodies comprised of one or more materials which are pharmaceutically acceptable. Such bodies can include liposomes formed of lipids, one of which is PG. Alternatively, the pharmaceutically acceptable bodies can be solid beads, hollow beads, filled beads, particles, granules and microspheres of biocompatible materials, which comprise one or one or more biocompatible materials such as polyethylene glycol, poly(methylacrylate), polyvinylpyrrolidone, polystyrene and a wide range of other natural, semi-synthetic and synthetic materials, with phosphate-glycerol groups attached thereto.

As noted above, analogues of phosphatidylglycerol with modified active head groups, which also interact with PG receptors on the antigen presenting cells, through the same receptor pathway as PG or otherwise resulting in a rapid anti-inflammatory reaction in the recipient body are contemplated within the scope of the term phosphatidylglycerol. This includes, without limitations compounds in which one or more of the hydroxyl groups and/or the phosphate group is derivatized, or in the form of a salt. Many such compounds form free hydroxyl groups in vivo, upon or subsequent to administration and, accordingly, comprise convertible phosphate-glycerol groups.

Preferred compositions of matter for use in the process of the invention are liposomes, which may be composed of a variety of lipids. Preferably, however, none of the lipids are positively charged. In the case of liposomes, phosphatidyl glycerol PG may constitute the major portion or the entire portion of the liposome layer(s) or wall(s), oriented so that the phosphate-glycerol head group portion thereof is presented exteriorly, to act as the binding group, and the lipid chain or chains form the structural wall.

Liposomes, or lipid vesicles, are sealed sacs, in the micron or sub-micron range, the walls (monolayer or multilayer) of which comprise suitable amphiphiles. They normally contain an aqueous medium, although for the present invention the interior contents are unimportant, and generally inactive. Accordingly, in a preferred embodiment, the liposomes, as well as other pharmaceutically acceptable bodies, are essentially free of non-lipid pharmaceutically active entities (e.g. <1%) and more preferably are free of non-lipid pharmaceutically active entities. Such liposomes are prepared and treated so that the active head groups are presented exteriorly on the liposomal body. The PG in the liposomes of the preferred embodiments of this invention thus serves as both a ligand and a structural component of the liposome itself.

Thus a preferred embodiment of this invention uses liposomal bodies which expose or can be treated or induced to expose, on their surfaces, one or more phosphate-glycerol head groups to act as binding groups. Phosphatidylglycerol should comprise from 10%-100% of the liposome, with the balance being an inactive constituent, e.g. phosphatidylcholine PC, or one which acts through a different mechanism, phosphatidylserine PS, or mixtures of such. Inactive co-constituents such as PC are preferred.

At least 10% by weight of such liposome is composed of PG, preferably from 50%-95%, more preferably from 60-90% and most preferably from 70-90%, with the single most preferred embodiment being about 75% by weight of PG, the balance preferably being PC.

Mixtures of PG liposomes with inactive liposomes and/or with liposomes of phospholipids acting through a different mechanism can also be used.

As regards non-liposomal bodies for use in the present invention, these as noted include biocompatible solid or hollow beads of appropriate size. The biocompatible non-liposomal synthetic or semi-synthetic bodies may be selected from polyethylene glycol, poly(methylmethacrylate), polyvinylpyrrolidone, polystyrene and a wide range of other natural, semi-synthetic and synthetic materials, with phosphate-glycerol groups attached to the surfaces thereof. Such materials include biodegradable polymers, such as disclosed by Dunn, et al. U.S. Pat. No. 4,938,763, which is hereby incorporated by reference in its entirety.

Biodegradable polymers are disclosed in the art and include, for example, linear-chain polymers such as polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, chitin, chitosan, and copolymers, terpolymers and combinations thereof. Other biodegradable polymers include, for example, gelatin, collagen, etc.

Suitable substances for derivatization to attach the phospholipid(s), or portions thereof with head groups or binding groups, to three-dimensional bodies are commercially available e.g. from Polysciences Inc., 400 Valley Road, Warrington, Pa. 18976, or from Sigma Aldrich Fine Chemicals. Methods for their derivatization are known in the art. Specific preferred examples of such methods are disclosed in International Patent Application PCT/CA02/01398 Vasogen Ireland Limited, which is incorporated herein by reference.

It is contemplated that the patient may be a mammal, including but not limited to humans and domestic animals such as cows, horses, pigs, dogs, cats and the like.

Phospholipids are amphiphilic molecules (i.e. amphiphiles), meaning that the compound comprises molecules having a polar water-soluble group attached to a water-insoluble hydrocarbon chain. The amphiphiles serving as the layers of the matrix have defined polar and apolar regions. The amphiphiles can include, in addition to PG for use in this invention, other lipids used alone with the phospholipid carrying the active head group, or in admixture with another. The amphiphiles serving as the layer(s) of the liposomes can be inert, structure-conferring synthetic compounds such as polyoxyethylene alkylethers, polyoxyethylene alkylesters and saccharosediesters.

Methods of preparing liposomes of the appropriate size are known in the art and do not form part of this invention. Reference may be made to various textbooks and literature articles on the subject, for example, the review article “Liposomes as Pharmaceutical Dosage Forms”, by Yechezkel Barenholz and Daan J. A. Chrommelin, and literature cited therein, for example New, R. C. “Liposomes: A Practical Approach”, IRL Press at Oxford University Press (1990).

The diameter of the liposomes, as well as the other pharmaceutically acceptable bodies, for use in the preferred embodiment of this invention is from about 20 nm to about 500 μm, more preferably from about 20 nm to about 1000 nm, more preferably from about 50 nm to about 500 nm, and most preferably from about 80 nm to about 120 nm (preferably measured along its longest axis). In one embodiment, the diameter of the liposome is from 60 nm to 500 μm.

The pharmaceutically acceptable bodies may be suspended in a pharmaceutically acceptable carrier, such as physiological sterile saline, sterile water, pyrogen-free water, isotonic saline, and phosphate buffer solutions (e.g. sterile aqueous solutions comprising phosphate buffer), as well as other non-toxic compatible substances used in pharmaceutical formulations, such as, for example, adjuvants, buffers, preservatives, and the like. Preferably, the pharmaceutically acceptable bodies are constituted into a liquid suspension in a sterile biocompatible liquid such as buffered saline and administered to the patient by any appropriate route which exposes it to one or more components of the immune-system, such as intra-arterially, intravenously or most preferably intramuscularly or subcutaneously.

It is contemplated that the pharmaceutically acceptable bodies may be freeze-dried or lyophilized so that they may be later resuspended for administration in the process of the invention. The lyophilized or freeze-dried binding group carrying bodies may include a pharmaceutically acceptable carrier, such as physiological sterile saline, sterile water, pyrogen-free water, isotonic saline, and phosphate buffer solutions (e.g. sterile aqueous solutions comprising phosphate buffer), as well as other non-toxic compatible substances used in pharmaceutical formulations, such as, for example, adjuvants, buffers, preservatives, and the like. Protectants for freeze drying, as known in the art, for example lactose or sucrose, may also be included.

A preferred manner of administering the pharmaceutically acceptable bodies to the patient is a course of injections, preferably intramuscular or subcutaneous, administered twice daily, daily, several times per week, weekly or monthly to the patient, over a period ranging from a few days to several weeks. The frequency and duration of the course of the administration is likely to vary from patient to patient, and according to the acute condition being treated and its severity. Its design and optimization is well within the skill of the attending physician. Intramuscular injection, especially via the gluteal muscle, is most preferred. One particular injection schedule, in at least some of the indications of the invention, is an injection, via the gluteal muscle, of an appropriate amount of bodies on day 1, a further injection on day 2, and a further injection on day 14, and then “booster” injections at monthly intervals, if appropriate to prevent recurrence of the acute condition.

It is postulated that, in many embodiments of the present invention, pharmaceutically acceptable bodies comprising the phosphate-glycerol head groups as binding groups on their surface are acting as modifiers of the patient's immune system, in a manner similar to that of a vaccine. Accordingly they are used in quantities and by administration methods to provide a sufficient localized concentration of the bodies at the site of introduction. Quantities of such bodies appropriate for immune system modification may not be directly correlated with body size of a recipient and can, therefore, be clearly distinguished from drug dosages, which are designed to provide therapeutic levels of active substances in a patient's bloodstream and tissues. Drug dosages are accordingly likely to be much larger than immune system modifying dosages.

The correlation between weights of liposomes and numbers of liposomes is derivable from the knowledge, accepted by persons skilled in the art of liposomal formulations, that a 100 nm diameter bilayer vesicle has 81,230 lipid molecules per vesicle, distributed approximately 50:50 between the layers (see Richard Harrigan—1992 University of British Columbia PhD Thesis “Transmembrane pH gradients in liposomes (microform): drug-vesicle interactions and proton flux”, published by National Library of Canada, Ottawa, Canada (1993); University Microfilms order no. UMI00406756; Canadiana no. 942042220, ISBN 0315796936). From this one can calculate, for example, that a dose of 5×10⁸ vesicles is equivalent to 4.06×10¹³ lipid molecules. Using Avogadro's number for the number of molecules of lipid in a gram molecule (mole), 6.023×10²³, one determines that this represents 6.74×10⁻¹¹ moles which, at a molecular weight of 729 for PG is approximately 4.92×10⁻⁸ gm, or 49.2 nanograms of PG for such dosage. For a dose of 6×10⁵ vesicles, of the order of the dose used in the specific in vivo examples below, the corresponding calculation gives a weight of 5.89×10⁻¹¹ gm, or 0.059 nanograms.

The quantities of the pharmaceutically acceptable bodies to be administered will vary depending on the nature of the acute inflammatory disorder it is intended to treat and on the identity and characteristics of the patient Preferably, the effective amount of pharmaceutically acceptable bodies is non-toxic to the patient, and is not so large as to overwhelm the immune system. When using intra-arterial, intravenous, subcutaneous or intramuscular administration of a sterile aqueous suspension of pharmaceutically acceptable bodies, it is preferred to administer, for each dose, from about 0.1-50 ml of liquid. Preferably, the number of bodies administered per delivery to a human patient is in the range from about 500 to about 2.5×10⁹ (<250 ng of bodies, in the case of liposomes, pro-rated for density differences for other embodiments of bodies), more preferably from about 1,000 to about 1,500,000,000, even more preferably 10,000 to about 100,000,000, and most preferably from about 200,000 to about 2,000,000.

Since the pharmaceutically acceptable bodies are believed to be acting, in the process of the invention, as immune system modifiers, in the nature of a vaccine, the number of such bodies administered to an injection site for each administration may be a more meaningful quantitation than the number or weight of bodies per unit of patient body weight. For the same reason, it is now contemplated that effective amounts or numbers of bodies for small animal use may not directly translate into effective amounts for larger mammals (i.e. greater than 5 kg) on a weight ratio basis.

The present invention is a process for the treatment of or prophylaxis against acute inflammatory mammalian disorders where inappropriate cytokine expression is involved. Those disorders are generally characterized by acute inflammation that is mediated by cytokines IL-1β, IFN-γ and/or cytokines secreted from inflammatory cells e.g. Th-1 cells. A patient having such a disorder may be selected for treatment.

“Treatment” includes, for example, a reduction in the number of symptoms, a decrease in the severity of at least one symptom of the particular disease or a delay in the further progression of at least one symptom of the particular disease.

One example of an acute inflammatory disorder that the process of the present invention may treat or help guard against, is acute allergic or toxic reaction from surface contact with environmental and occupational allergens or drugs through anaphylactic shock. More specific examples of such disorders include allergic contact dermatitis, acute hypersensitivity and respiratory allergy.

A second example of an acute inflammatory disorder that the process of the present invention may treat or help guard against, is acute neurological inflammatory injury such as that caused by acute infection.

A third example of an acute inflammatory disorder that the process of the present invention may treat or help guard against, is acute myocardial infarction.

Another example is prophylaxis against or treatment of acute neuronal injury resulting from cardiopulmonary bypass surgery.

The invention may also be useful in pre-conditioning individuals about to enter an environment in which they will encounter conditions likely to lead to acute inflammatory disorder development, such as harmful chemical-containing environments and insect infested areas.

The prophylaxis or treatment methods described herein may be administered in combination with one or more other modalities. Examples of other preferred modalities include, but are not limited to, non-steroidal and steroidal anti-inflammatories. Administration in combination includes, for example, administration of the compositions described herein, prior to, during or after administration of the other one or more modalities. One of skill in the art will be able to determine the administration schedule and dosage.

### EXAMPLE 1

Liposomes of 100±20 nm in average diameter and comprising 25% by weight phosphatidylcholine and 75% by weight PG (phosphatidylglycerol) were prepared according to standard methods known in the art. A stock suspension of liposome composition containing 4.8×10¹⁴ liposomes per ml was diluted with PBS to give an injection suspension containing 6×10⁵ liposomes per 50 microlitres. The liposomai suspensions were injected into female BALB/c mice (Jackson Laboratories) aged 6-8 weeks and weighing 19-23 g, to determine the effect on cytokine modulation at the lymph nodes, in a murine, acute dinitrofluorobenzene (DNFB) induced inflammatory model.

The animals were assigned to one of 2 groups, A and B, with 20 animals in each group. Group A was a positive control group, receiving a 50 microlitre injection of PBS and DNFB irritant treatment, but no liposomes. Group B was treated with DNFB and received an injection of 50 microlitres of PBS containing approximately 6×10⁵ of the above-identified liposomes.

Immediately prior to the injections, animals of Groups A and B were anaesthetized with 0.2 ml 5 mg/ml sodium pentobarbital via IP injection. The abdominal skin of the mouse was sprayed with 70% EtOH and a scalpel blade was used to remove about a one-inch diameter patch of hair from the abdomen. The shaved area was then painted with 25 μl of 0.5% 2,4-dinitrofluorobenzene (DNFB) in 4:1 acetone:olive oil using a pipette tip.

The products were administered by injection into the lateral gastrocnemius muscle (right leg). Four animals from each group were sacrificed two hours after injection, four more after 6 hours, four more after 24 hours and the remaining four after 48 hours. From each sacrificed animal, the draining inguinal lymph node, from the same side as the injection, was harvested. The RNA was extracted from the lymph nodes, and subjected to RT-PCR analysis for expression of the pro-inflammatory cytokines TNF-α, IFN-γ and IL-12, and the anti-inflammatory cytokines TGF-β. The results were determined in comparison with the standard reporter gene GAPDH, which is known to be expressed at 100% levels.

The data, as cytokine/GAPDH for the various cytokines against time, are presented graphically on the accompanying Figures.

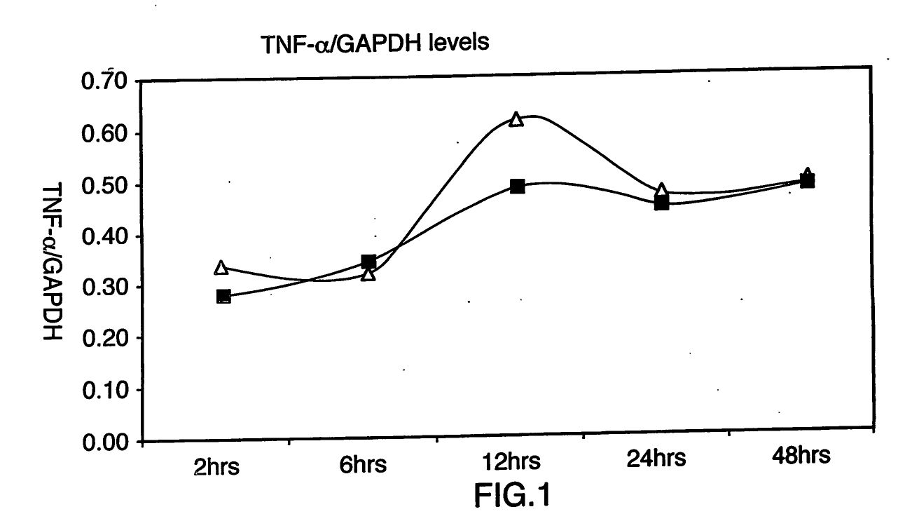

FIG. 1 pertains to TNF-α measurements. These are plotted, as a ratio to housekeeping gene GAPDH, as vertical axis, against time, with points at time 2 hours, 6 hours, 12 hours, 24 hours and 48 hours. Bach point represents the mean of four measurements. The curve with points represented by squares is derived from animals of Group B, i.e. treated with irritant and injected with liposomes, in accordance with the preferred embodiment of the invention. It is significantly lower, even at two hours, and even more markedly at 12 hours (p=0.0001) than the curve with triangular points, derived from animals of Group A, which received the irritant and PBS without liposomes. This shows the pro-inflammatory cytokine TNF-α, upregulated as a result of the administration of the DNFB, is rapidly downregulated by the liposomes. This is an indication of the potential of the process of the present invention to combat acute TNF-α related disorders in mammalian patients.

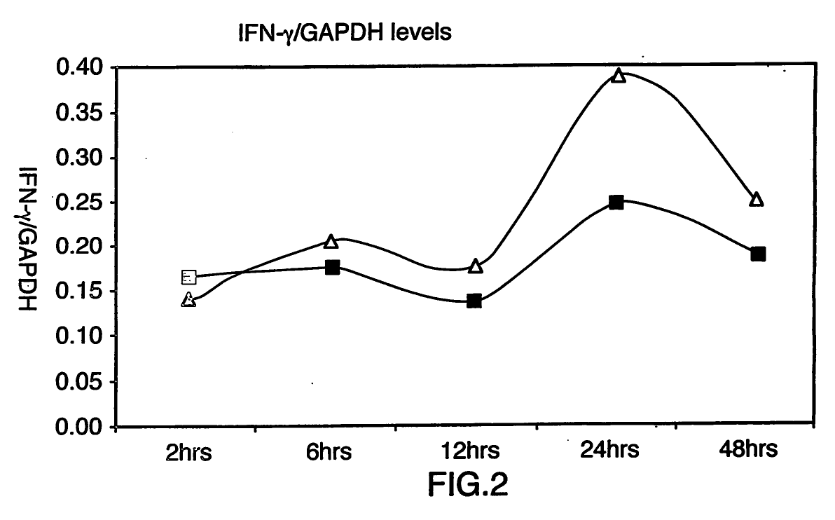

FIG. 2 similarly presents the results of measurements of IFN-γ, another pro-inflammatory cytokine. Here the effect of the liposomal formulation is noticeable and significant at 6 hours, and becomes even more pronounced at 24 hours (=0.002) and 48 hours (p=0.011), further indication of the potential of this invention in treating acute inflammatory disorders, especially those in which IFN-γ plays a significant role.

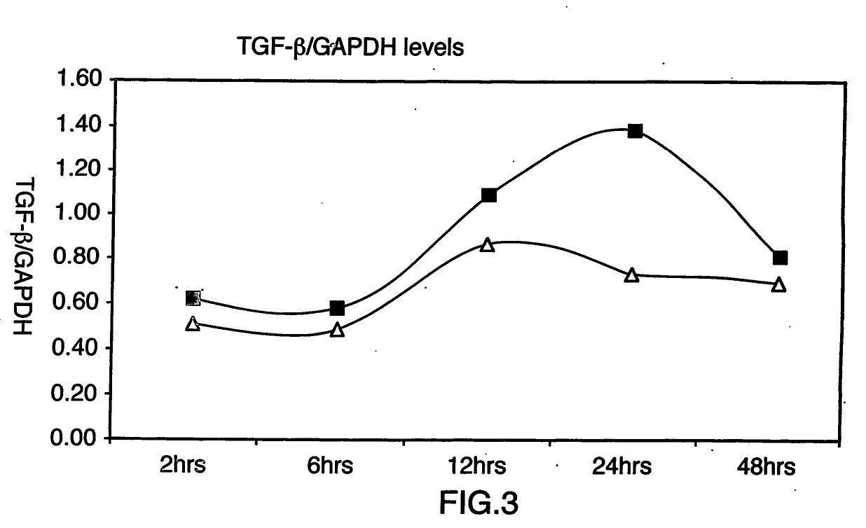

FIG. 3 similarly presents the results of measurements of TGF-β, an anti-inflammatory cytokine. The curve for animals of Group B, receiving both the irritant and the liposomes to combat the effects of the irritant is consistently above that for the Group A animals which received the irritant but no liposomes. At 12 and 24 hours, there is a large increase of TGF-β, as compared with the Group A animals' results (at 24 hours, p=0.001), clearly indicating the potential for the treatment according to the preferred process of the invention in treating acute inflammatory disorders.

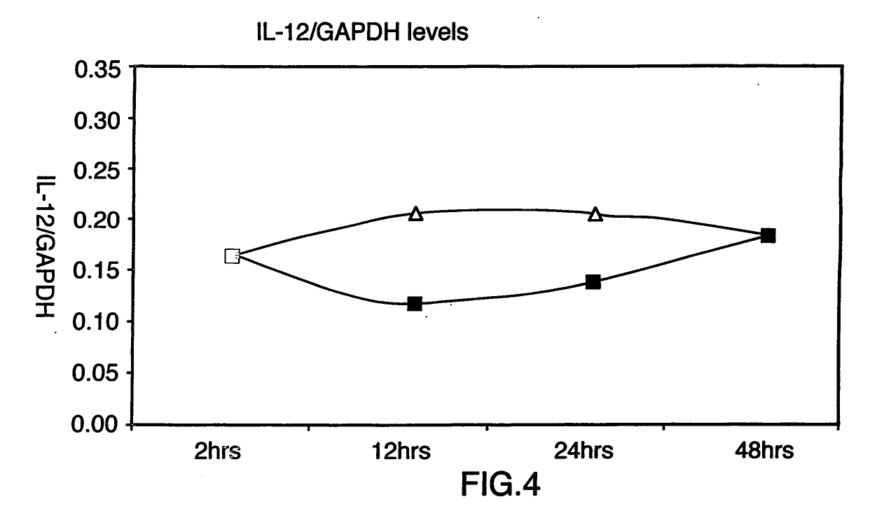

FIG. 4 similarly presents the results for measurement of IL-12, an inflammatory cytokine. Here, the reverse effect is observed, as compared with FIG. 3. The curve for the animals of Group B is consistently below that for the animals of Group A (at 12 hours, p=0.001; at 24 hours, p=0.042), indicating inhibition or down-regulation of this pro-inflammatory cytokine over the 12-48 hour period of measurement.

### EXAMPLE 2

U937 is a monocytic leukemia cell line that can be differentiated into macrophages by administration of a phorbol ester. Treatment of these macrophages with lipopolysaccharide (LPS), a component of the cell wall of gram-negative bacteria, stimulates an inflammatory response. Assessment of this inflammatory response by. measurement of inflammatory or anti-inflammatory cytokines, in vitro, and the effect of administering test substances on this response provides a measure of the anti-inflammatory properties of the test substances.

U937 cells were cultured by growing in RPMI medium with 10% fetal, serum and 1% periicillin/streptomycin at 37° C., 5% CO². They were seeded into six. well plates at a concentration of 5×10⁵ cells per ml. They were differentiated into macrophages by treating with 150 nM phorbol myristate acetate (PMA) for 2-3 days. The cell media was replaced, after the macrophages have differentiated, and replaced with complete media for 24 hours prior to liposome addition, so as to allow any upregulation of genes/proteins induced by PMA to be reduced.

Liposomes of standard size 100±20 nm were prepared according to standard methods known in the art, with one set comprising 75% phosphatidyl glycerol (PG), 25% phosphatidylcholine (PC) and the other comprising 100% PC. A stock concentration of 2.93×10¹⁴ liposomes per ml was used. This was diluted in PBS to a working concentration of 2.93×10⁸ liposomes per ml.

Differentiated U937 macrophages were treated with a dose range of PG/PC liposomes, in the presence and absence of LPS (10 ng/ml), and others were treated with a similar dose range of PC liposomes, in the presence and absence of the same amount of LPS. After 18 hours, cell supernatant was collected, frozen and subsequently analyzed for TNF-α. Measurement of TNF-α was carried out by Quantikine Elisa kits purchased from R&D systems.

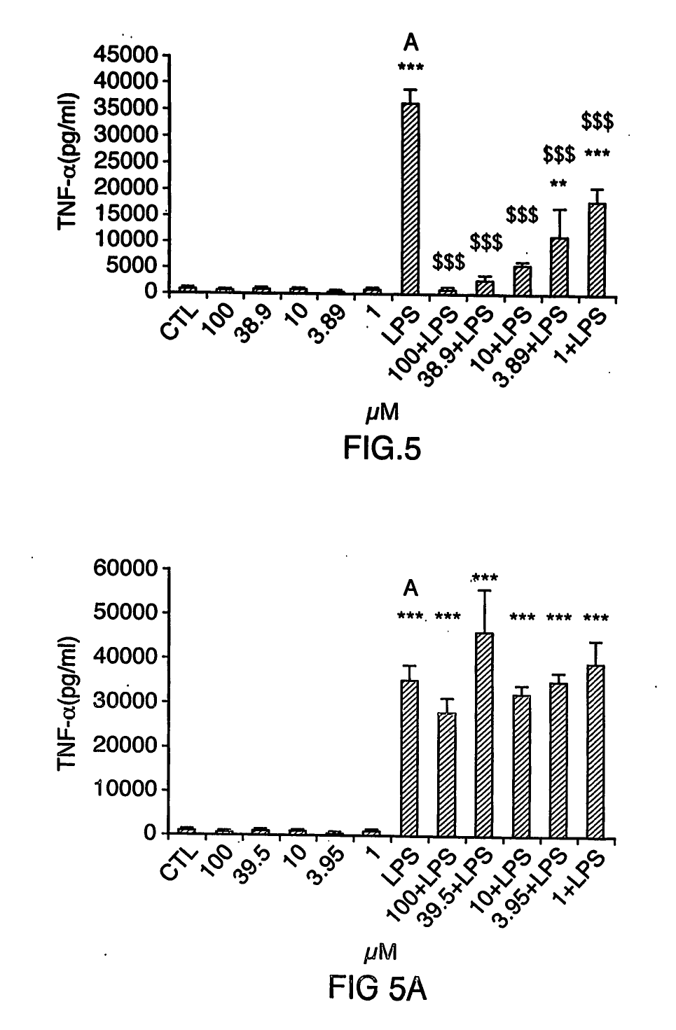

FIG. 5 of the accompanying drawings is a bar graph of the results obtained using the PO/PC liposomes and various dosages. The vertical axis is the amount of TNF-α. In picagrams per ml. Control experiments with liposomes in the absence of LPS showed no TNF-α content. Bar A Is a control experiment administering LPS alone. The other bars show the results of various micromolar concentrations of stock suspension of liposomes administered to the cells along with LPS. The results indicate a significant reduction in inflammatory cytokine TNF-α after 18 hours, in this model of acute inflammation, indicating utility of these liposomes in treatment of acute inflammatory conditions of the skin, derived from allergic reactions. FIG. 5A of the accompanying drawings similarly presents the results of the experiments using PC liposomes, and indicating a much lower, if any, reduction in inflammatory cytokine production by these liposomes. In all cases, the data are the means of four separate experiments.

### EXAMPLE 3

Male Wistar rats (bioresources unit, Trinity College, Dublin, Ireland) of mean age 4 months were used in these experiments. Animals were housed in groups of four to six under 12 of light schedule; ambient temperature was controlled between 22 and 23° C. rats were maintained under veteran Ray supervision throughout the study. The experiments were performed under license issued by the Department of Health and Children (Ireland).

Rats were randomly assigned to four treatment groups. Rats in two of these groups were injected with PG/PC liposomes as used in Example 3, 150 microlitres of the six times 10 to the sixth particles per mil suspension in PBS, intramuscularly into the upper hind limb, 14 days, 13 days and 24 hours before anesthesia. Groups of control rats were similarly injected with saline. Anesthesia was effected by intraperitoneal injection of urethane, 1.5 g per kilogram. The absence of a pedal reflects was considered to be an indicator of deep anesthesia. After anesthesia had taken full effect, one group of liposome—treated and one group of saline—treated rats were given an intraperitoneal injection of LPS (100 micrograms per kilogram) and the remaining two groups received saline intraperitoneally.

Approximately six hours after the anesthesia, rats were sacrificed by decapitation and the brains were rapidly removed. The hippocampus was dissected free from whole brain; cross-chopped slices (350 micrometers square) were prepared using a McIlwain tissue chopper and stored in Krebs buffer containing calcium chloride and 10% DMSO at −80° C. as previously described (Haan, E. A. and Bowen, D. M., J. Neurochem. 37,243-246) until required for analysis.

IL-4 concentration was assessed in hippocampal homogenates. Analysis was carried out by ELISA (R&D) Systems. Hippocampal slices were thawed, and rinsed three times in ice cold Krebs solution. Protein concentrations in homogenates were equalized (Bradford, M. M., 1976, Anal. Biochem. 72, 248-254), and triplicate aliquots (100 μl) were used by ELISA. Values were corrected for protein concentration in homogenate samples and values were expressed as picagrams per milligram protein.

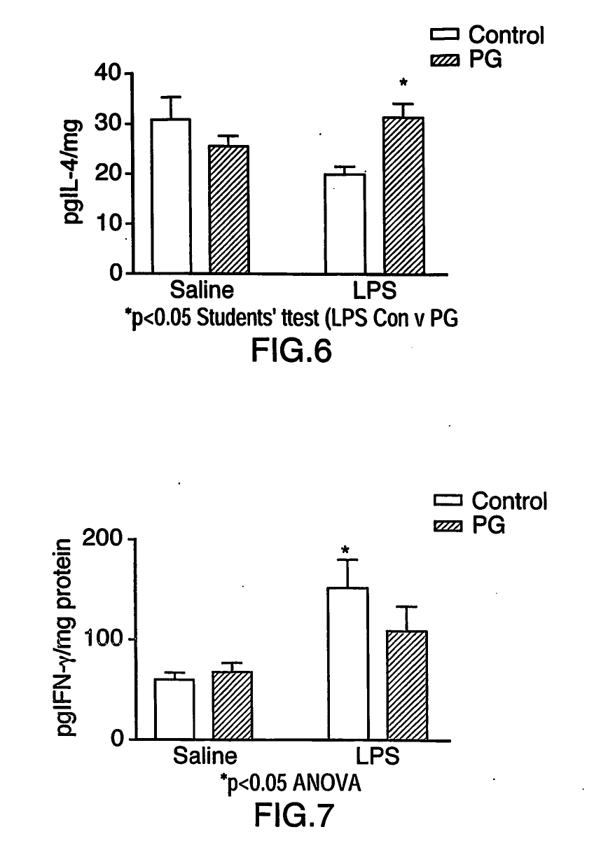

FIG. 6 of the accompanying drawings graphically presents the results for analysis of IL-4, an anti-inflammatory cytokine. A significant increase in IL-4 concentration is to be observed in the hippocacampal extracts from LPS treated rats which had received the pre-injections of liposomes, as compared with the saline controlled, LPS treated rats. This is an indication for use of the invention in prevention or treatement of acute inflammatory conditions of the hippocampus, such as those resulting from Ischemic injury to the brain.

In physiological systems, an upregulation of the anti-inflammatory cytokine IL-4 correlates with a down regulation of theinflammatory cytokine IL-1β. (See for example Goletti D, Kinter A L, Coccia E M, Battistini A, Petrosillo N, Ippolito G and Poli G, Cytokine, 2002 Jan. 7; 17(1): 28-35.

### EXAMPLE 4

40 male Wistar rats were allocated to one of four groups. One group received saline treatment only, the second group received liposomes only, the third group received LPS only, and the fourth group received LPS and liposomes. Injections were made intraperitoneally, using the same quantities of the respective materials as described in Example 3. The injections of liposomes in the fourth group took place one hour prior to the injection of LPS. The rats were returned to the home cages fully conscious. Rats were sacrificed six hour later, trunk blood was collected, and serum prepared. Serum was analyzed for IFN-γ content by ELISA (R&D Systems) using know, standard techniques.

The results of the measurement of IFN-γ in the serum are graphically presented on FIG. 7 of the accompanying drawings. A significant decrease in the concentration of IFN-γ in the LPS-treated groups which were pretreated with liposomes according to the present invention is to be noted, in the serum after six hours. This is an indication of the potential use of the present invention in prophylaxis or treatment of systemic acute inflammatory conditions.

1. A method of preventing or treating an acute inflammatory disorder in a mammalian patient of comprising administering an effective amount of pharmaceutically acceptable bodies carrying an effective number of phosphate-containing groups presented or presentable on the surface of said bodies, the phosphate-containing groups comprising a plurality of phosphate-glycerol groups or groups convertible to such groups, said bodies being of a size from about 20 nanometers(nm) to 500 micrometers(μm).

2. The method of claim 1, wherein the acute inflammatory disorder features an upregulation of at least one pro-inflammatory cytokine.

3. The method of claim 2, wherein the pro-inflammatory cytokine is selected from TNF-α, INFγ, IL-1 and IL-12.

4. The method of claim 1, wherein the acute inflammatory disorder features a downregulation of at least one anti-inflammatory cytokine

5. The method of claim 4, wherein the anti-inflammatory cytokine is selected from TGF-β, IL-10 and IL-4.

6. The method of claim 5, wherein the anti-inflammatory cytokine is TGF-β.

7. The method of claim 1, wherein the bodies are essentially free of pharmaceutically active entities other than phosphate-containing surface groups.

8. The method of claim 1, wherein the phosphate-glycerol groups constitute 60%-100% of the phosphate-containing surface groups on the bodies.

9. The method of claim 1, wherein the phosphate-glycerol groups correspond to the formula: —O—P(═O)(OH)—O—CH2-CH(OH) —CH2-OH

10. The method of claim 1, wherein the bodies are liposomes constituted to the extent of 60-100% by weight of a phosphatidyl glycerol phospholipid corresponding to the formula: 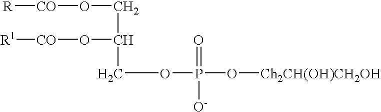
where R and R¹ are independently selected from C₁-C₂₄ hydrocarbon chains, saturated or unsaturated, straight chain or containing a limited amount of branching wherein at least one chain has from 10 to 24 carbon atoms.

11. The method of claim 1, wherein the acute inflammatory disorder is acute allergic or toxic reaction from surface contact with environmental allergen or drugs through anaphylactic shock.

12. The method of claim 11, wherein the acute inflammatory disorder is allergic contact dermatitis or acute hypersensitivity.

13. The method of claim 1, wherein the acute inflammatory disorder is acute neurological inflammatory injury.

14. The method of claim 1, wherein the acute inflammatory disorder is acute neuronal injury resulting from cardiopulmonary bypass surgery.

15. The method of claim 1, wherein preventing or treating an acute inflammatory disorder comprises inhibiting and/or reducing the progression of the acute inflammatory disorder
